(19) United States
(12) Patent Application Publication
(10) Pub. No.: US 2006/0287728 A1
(43) Pub. Date: Dec. 21, 2006

(54) System and method for implanting intervertebral disk prostheses

(76) Inventors: Mourad Ben Mokhtar, Paris (FR); Olivier Carli, Geneva (CH)

Correspondence Address: CARL M. NAPOLITANO, PH.D.;ALLEN, DYER, DOPPELT, MILBRATH & GILCHRIST, P.A., 255 SOUTH ORANGE AVE., SUITE 1401, P.O. BOX 3791, ORLANDO, FL, 32802-3791, US

(21) Appl. No.: 11/158,273

(22) Filed: Jun. 21, 2005

(51) Int. Cl. A61F 2/44; A61F 2/46; A61B 17/88

(52) U.S. Cl. 623/17.14; 606/90

(57) ABSTRACT

A system for implanting a disk prosthesis within a cervical spine includes a disk prosthesis holder having tines for frictionally engaging the disk prosthesis through an anterior to posterior movement, and a cervical disk distractor having jaws with guide rails for freely receiving the disk prosthesis. Inserting the cervical distractor while in a closed position into the intervertebral space allows an opening of the jaws for increasing a separation between opposing vertebrae. Inserting the disk prosthesis between the opened jaws using the cervical disk holder allows for a placing of the disk prosthesis within the cavity of the intervertebral space with an aligning of the disk prosthesis. Closing the jaws permits a removing of the cervical disk holder from the disk prosthesis with a subsequent removing of the cervical distractor resulting in an implanting of the disk prosthesis within the intervertebral space.

### FIELD OF INVENTION

The present invention generally relates to spinal implants for use in intervertebral disk replacement, and more particularly to an articulating disk prostheses and insertion device for artificially replacing the fibro-cartilaginous disk that connects vertebrae of a spinal column.

### BACKGROUND

Intervertebral disks comprising a deformable element, known as nucleus pulposus, surrounded by a number of elastic fibrous layers, can undergo alterations such as compression, deformation, slippage or wear and, more generally, degeneration associated with mechanical stresses applied to it. This may result in anatomical and functional destruction of the disk and of the vertebral segment. This alteration of the disk changes the mechanical behavior of the intervertebral disk and leads to a reduction in the height of the intersomatic space, which results in a disturbance of articular function as a whole. This produces instability that, in particular, creates an osteoarthritic reaction that is the source of pain and of osteophytic processes.

It is well known to replace a defective disk with an artificial disk, designed so as to attempt to reproduce the kinematics of a natural movement. Typically, implanting a disk prosthesis is relatively traumatizing to the vertebral plates and does not make it possible to ensure an optimum transfer of the thrust of an upper vertebra on an adjoining lower vertebra. Further, the contact interface between typically used polyethylene cores and titanium plates degrades over time, thus undesirably modifying the mobility of the prosthesis.

The present invention remedies such drawbacks by providing for an effective insertion of an intervertebral disk prosthesis that faithfully reproduces natural movements of the intervertebral disk and provides a desirable transmission of the thrust of upper vertebrae on lower vertebrae while offering control of the relative angular clearance between the vertebrae.

### SUMMARY

A spinal disk prosthesis restores normal physiological function in the spine by preserving intervertebral motion, stability, lordosis, and spacing while protecting vascular, neural, and other spinal structures. The present invention is directed to a system for implanting a disk prosthesis. One system for implanting a disk prosthesis within a cervical spine of a patient may include using a flat distractor for sizing a cavity formed within an intervertebral disk within an intervertebral space between adjacent upper and lower vertebrae. A trial disk implant may then be inserted into the cavity and removed once a desirable fit for the disk prosthesis is anticipated. A disk prosthesis holder may be employed to frictionally engage the disk prosthesis for inserting the disk prosthesis between the vertebrae. A disk distractor may also be provided with jaws for inserting the jaws between the adjacent vertebrae for distracting the adjacent vertebrae by moving the jaws from a closed position to an open position and for guiding the disk prosthesis in combination with the disk prosthesis holder into the intervertebral space. Once the disk prosthesis is desirably positioned within the cavity, the disk distractor is positioned from the open position to the closed position for permitting the adjacent vertebrae to be biased against the disk prosthesis. The disk prosthesis holder is pulled away from the disk distractor while permitting the disk prosthesis to remain in place between the adjacent vertebrae. The disk distractor may then be removed leaving the implanted disk prosthesis as desired.

### BRIEF DESCRIPTION OF DRAWINGS

For a fuller understanding of the invention, reference is made to the following detailed description, taken in connection with the accompanying drawings illustrating various embodiments of the present invention, in which:

FIG. 1 is a partial perspective view of one embodiment of a disk prosthesis implanted within a cavity formed within the intervertebral space between adjacent cervical disks;

FIG. 1A is a perspective view of a system of the present invention illustrating embodiments of a disk prosthesis and instruments for an implanting thereof in keeping with the present invention;

FIGS. 2 and 2A are perspective views illustrating one embodiment of a flat distractor useful in sizing a cavity to be formed within disk material between adjacent cervical disks;

FIGS. 3 and 3A are perspective views illustrating one embodiment of a disk distractor operable with a disk holder in keeping with the teachings of the present invention;

FIGS. 4 and 4A are perspective and side views of a trial implant device in keeping with the teachings of the present invention;

FIG. 4B is a perspective view of the trial implant of FIG. 4 illustrating one operable use within a cavity formed in an intervertebral space between adjacent vertebrae;

FIG. 5 is a front view in the frontal plane of a first exemplified embodiment of prosthesis according to the invention in a median position;

FIG. 6 is a cutaway view in the frontal plane of the prosthesis according to the invention, in an inclined position;

FIG. 7 is a side view in the sagittal plane of the prosthesis according to the invention in a median position;

FIG. 8 is a cutaway view in the sagittal plane of prosthesis according to the invention in an inclined position;

FIG. 9 is a top perspective in the horizontal or coronal plane of the prosthesis according to the invention;

FIGS. 10 and 11 are cutaway views in the frontal plane and in the sagittal plane, respectively, of a second exemplified embodiment of prosthesis according to the invention in an inclined position;

FIGS. 12 and 13 are cutaway views in the frontal plane and in the sagittal plane, respectively, of a third exemplified embodiment of the prosthesis according to the invention in an inclined position;

FIGS. 14, 14A, and 15 are front, front perspective, and side views of an alternate embodiment of the disk prosthesis of FIG. 5;

FIG. 16 is a cross sectional view taken through lines 12-12 of FIG. 10;

FIGS. 17 and 18 are front and side views of an alternate embodiment of the disk prosthesis of FIG. 14A;

FIG. 19 is a cross sectional view taken through lines 115-115 of FIG. 17;

FIG. 20 is a perspective view of a flat distractor in keeping with the teachings of the present invention;

FIGS. 21, 22, and 23 are front, side, and top views, respectively, of a head portion of the trial implant of FIG. 4;

FIG. 24 is a perspective view for one embodiment of a disk prosthesis holder in keeping with the teachings of the present invention;

FIG. 25 is a partial enlarged view of a head portion of the disk prosthesis holder of FIG. 24;

FIG. 26 is a partial perspective view of the disk prosthesis holder frictionally engaging the disk prosthesis of FIG. 14A;

FIGS. 26A and 26B are partial enlarged perspective and top views, respectively, of a head portion of the disk prosthesis holder of FIG. 26;

FIGS. 27, 27A, and 27B are rear, top, and bottom perspective views, respectively, for one embodiment of a cervical disk distractor in keeping with the teachings of the present invention;

FIGS. 28 and 28A are partial perspective and end views of a jaws portion of the distractor of FIG. 27 illustrating jaws in a closed position; and

FIGS. 29 and 29A are partial perspective and end views of the jaws portion of the distractor of FIG. 27 illustrating the jaws in an open position.

### DETAILED DESCRIPTION OF EMBODIMENTS

The present invention will now be described more fully with reference to the accompanying drawings in which alternate embodiments of the invention are shown and described. It is to be understood that the invention may be embodied in many different forms and should not be construed as limited to the illustrated embodiments set forth herein. Rather, these embodiments are provided so that this disclosure may be thorough and complete, and will convey the scope of the invention to those skilled in the art.

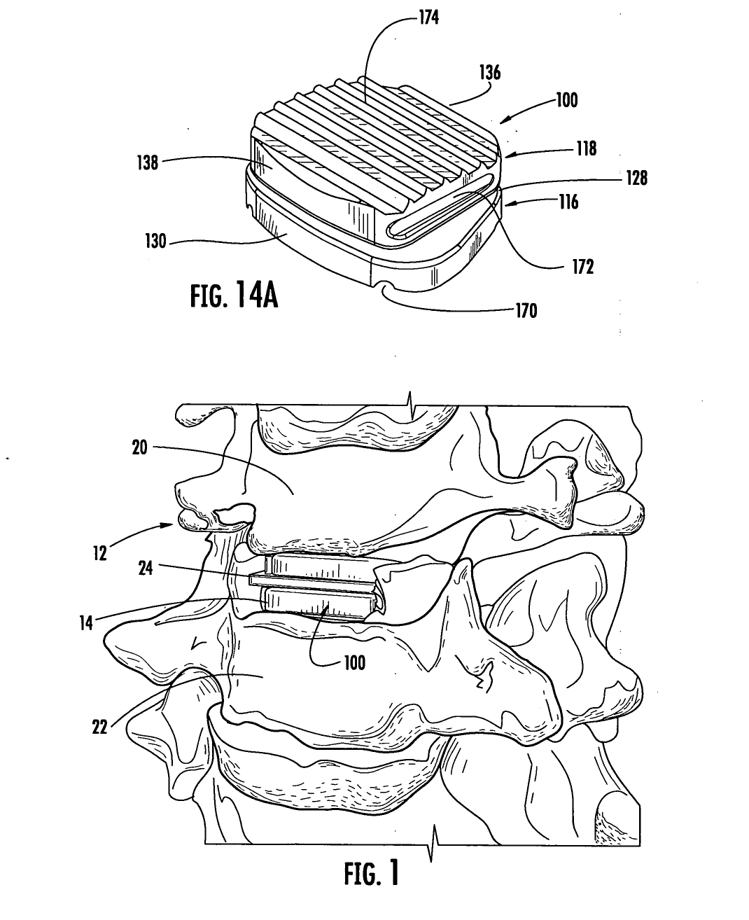

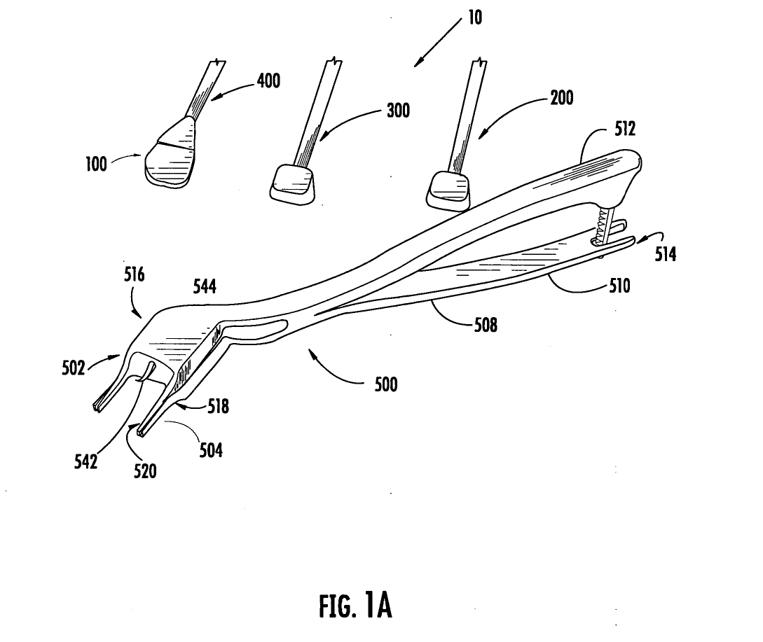

With reference initially to FIGS. 1-4, a system 10 for implanting a disk prosthesis 100 within a cervical spine 12 of a patient may include using a flat distractor 200 for sizing a cavity 14 formed within an intervertebral disk 16 within an intervertebral space 18 between adjacent upper and lower vertebrae 20, 22. A trial disk implant 300 is then inserted into the cavity 14 and removed once a desirable fit for the disk prosthesis 100 is anticipated. A disk prosthesis holder 400 frictionally engages the disk prosthesis 100 for inserting the disk prosthesis between the vertebrae 20, 22. A disk distractor 500 is provided with jaws 502 which are inserted between the adjacent vertebrae 20, 22 for distracting the adjacent vertebrae by moving the jaws from a closed position 504 to an open position 506 and for guiding the disk prosthesis 100 in combination with the disk prosthesis holder 400 into the intervertebral space 18. Once the disk prosthesis 100 is desirably positioned within the cavity 14, the disk distractor 500 is adjusted from the open position 506 to the closed position 504 for permitting the adjacent vertebrae 20, 22 to be biased against the disk prosthesis 100. The disk prosthesis holder 400 is pulled away from the disk distractor 500 while permitting the disk prosthesis 100 to remain in place between the vertebrae 20, 22. The disk distractor 500 is then removed leaving the implanted disk prosthesis 100 as desired.

Generally, preparing a patient for implanting a cervical spine prosthesis may include applying general anesthesia with an endotra-cheal intubation of the patient, affixing the head of the patient in a neutral position with an angle of rotation of approximately 30° for limiting tension applied to a sterno-cleido-mastoid muscle, lowering the shoulders of the patient, and fastening the shoulders to the surface for clearing the inferior cervical spine. A skin incision is made on a lesion level for vertebral disks to be processed, and the incised skin is drawn back typically using image intensification for exposing disk material.

As will become obvious to those skilled in the art, various prostheses and vertebrae may be applicable, as desired. By way of example, and with reference to FIGS. 5-8, one embodiment of the disk prosthesis 100 may be described as including a first plate 116 illustrated as a lower plate in the illustrated example, and a second plate 118 illustrated as an upper plate. The plates 116, 118 are intended to be attached to the adjacent vertebrae 20, 22 and are herein described, by way of example, as each having an outer face, 120, 122, respectively, and each having an inner face 124, 126, respectively, extending opposite one another. By way of example, the plates 116, 118 may be made of titanium or a titanium alloy. In one embodiment, the first and second plates comprise titanium with having an hydroxy-apatite coating.

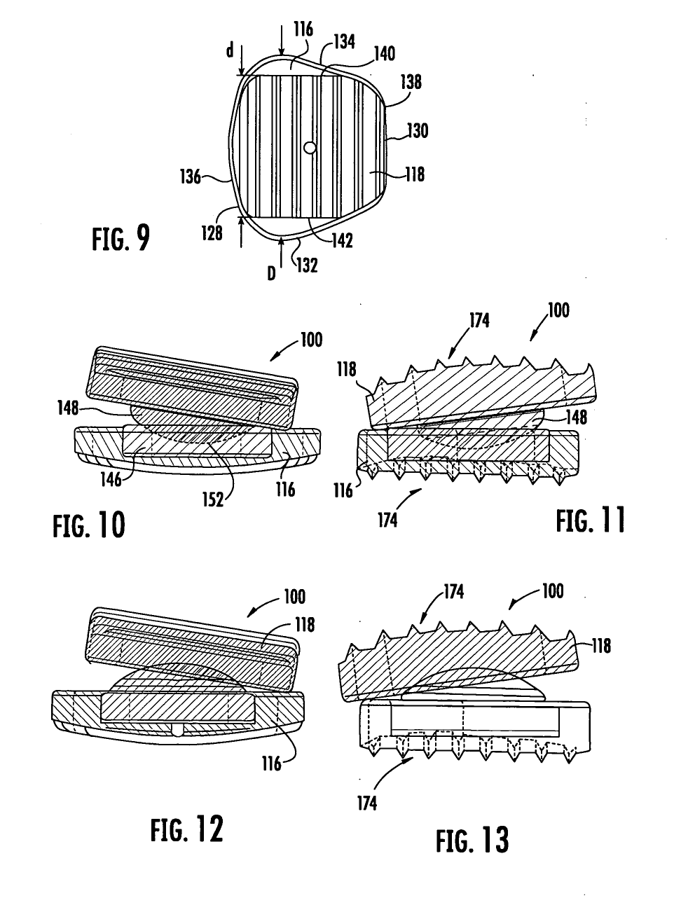

With continued reference to FIG. 5, and to FIG. 9, the first plate 116, also known as a carrier plate, has a transversal dimension D greater than the corresponding dimension d of the second plate 118. These dimensions d, D are measured in a transversal direction at the intersection between the horizontal plane T, illustrated with reference again to FIG. 9, and the frontal plane F, illustrated with reference again to FIG. 5, and are herein considered in terms of well known anatomical planes.

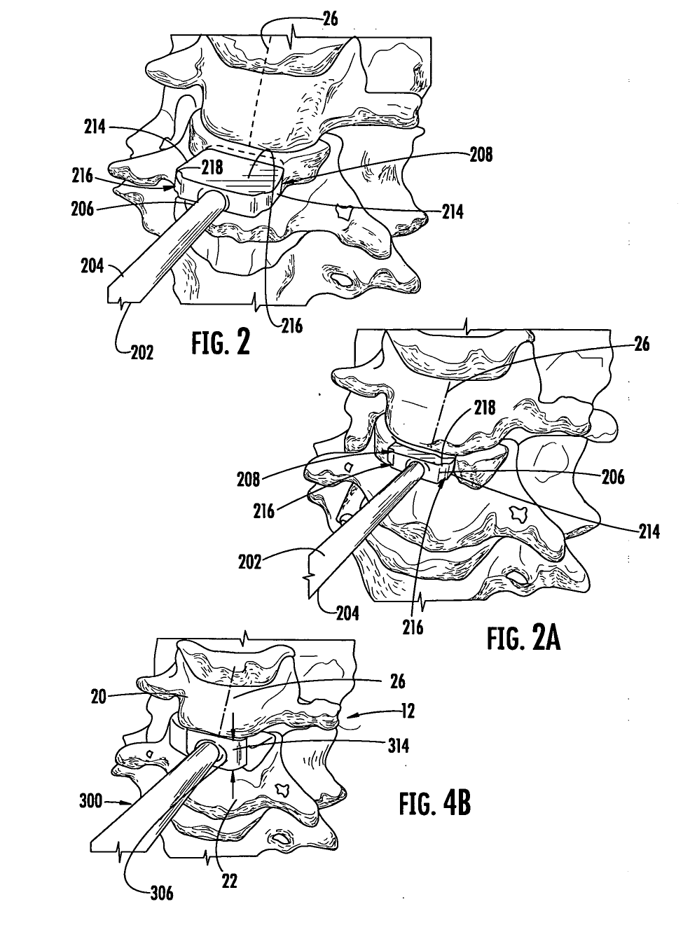

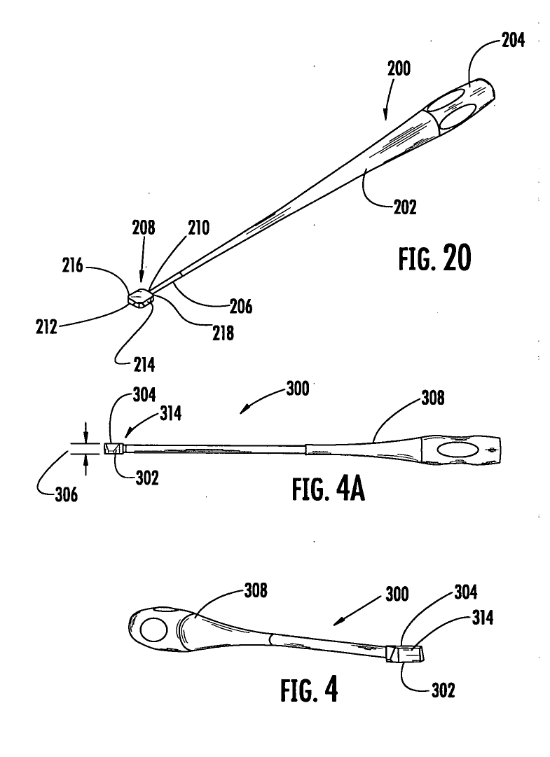

With reference to FIGS. 2 and 4, the inner face 124 of the first plate 116 thus constitutes a limit stop for the second plate 118, and with the outer face 120 of the first plate 116, a bearing surface for the vertebra 20 that is larger than the surface of the vertebra 22 contacted by the outer surface 122 presented by the second plate 118. As illustrated with reference again to FIG. 9, the first plate 116, the bearing plate, may have in the horizontal plane T, a substantially trapezoidal shape whose large base defines a leading edge 128, while a small base defines a trailing edge 130. The training edge 130 is connected to the leading edge 128 by opposing divergent edges 132, 134. Connecting fillets may preferably connect the leading and trailing edges 128, 130 to the divergent edges 132, 134.

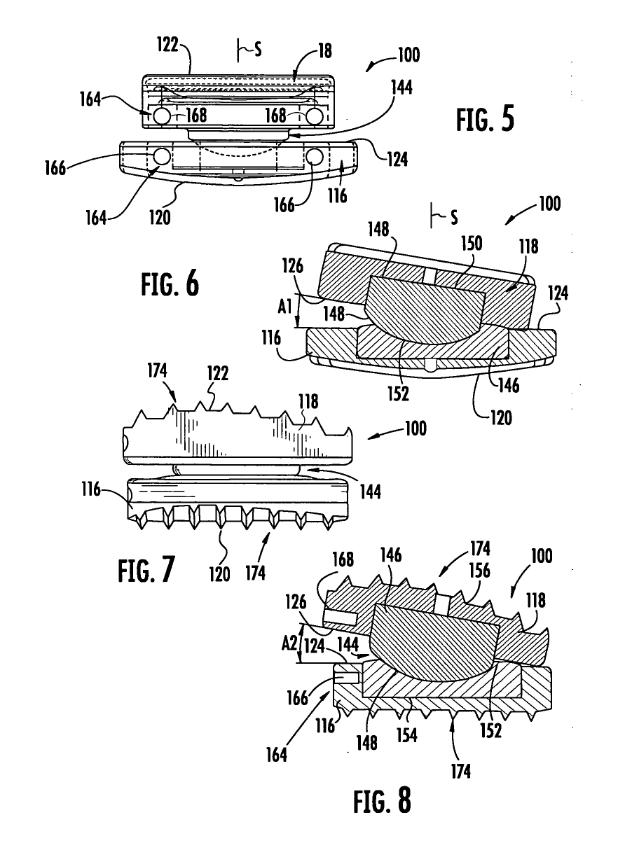

As illustrated with reference again to FIGS. 5 and 6 for the one embodiment herein described by way of example, the first plate 116 has the inner face 124 with a flat profile and the outer face 120 with a convex profile in the frontal plane F. With reference again to FIG. 5, the second plate 118 is, in the horizontal plane T, substantially the shape of a parallelepiped, with a leading edge 136 and a trailing edge 138 that are substantially parallel and merged in a stacked position with the leading edge 128 and the trailing edge 130 of the first plate 116. The leading edge 136 of the second plate 118 is connected to the trailing edge 138 through two linking edges 140, 142 substantially parallel to one another. Connecting fillets may connect the leading and trailing edges 136, 138 to the linking edges 140, 142. As is clearly shown with reference again to FIGS. 5, 6, and 9, the first plate 116 thus juts out in the horizontal plane T on either side of the linking edges 140, 142 of the second plate 118.

With continued reference to FIGS. 5-8, the discal prosthesis 100 herein described includes a ball-and-socket joint 144 interposed between the two plates 116, 118 that are stacked one on top of the other. In the exemplified embodiment illustrated in FIGS. 5 to 9, the ball-and-socket joint 144 comprises cooperating arcuate surfaces which mat include a first insert 146 that has a spherical cap 148 and a second insert 150 that has a spherical cup 152 that smoothly works with the spherical cap 148. Each insert 148, 150 is mounted in a hole 154, 156, a blind hole for example, through the inner face 124, 126 of each plate 116, 118. Each insert 146, 150 has a general form of revolution and has, respectively, a base 158, 160 of circular transversal cross-section, one of whose ends is arranged so as to have either the spherical cap 148 or the spherical cup 152. By way of example, the inserts 146, 150 may be made of a ceramic material or of polyethylene.

For the disk prosthesis 100 herein described by way of example, the first plate 116, the carrier plate, is wider than the second plate 118 in the transverse direction so as to constitute, on the one hand, the flat limiting surface, the inner surface 124, for the second plate 118 and, on the other hand, a large bearing surface, the outer face 120, with the associated vertebra. As is further clear with reference again to FIGS. 6 and 8, the prosthesis limit positions, both in the frontal plane F and in the sagittal plane S, are determined by bringing the plates 116, 118 into contact with one another.

As further illustrated with reference again to FIGS. 5-9, one embodiment of the disk prosthesis 100 may be dimensioned in such a manner that: E/d=ED₁ for A₁/A₂>0.5, wherein E represents the distance between the two inner faces 124, 126 of the plates 116, 118; d represents the width of the second (upper) plate 118 in the frontal plane F; D₁ represents the length of the second (upper) plate 18 in the sagittal plane S; A₁ represents the maximum angle of clearance between the plates 116, 118 in the frontal plane F; and A₂ represents the maximum angle of clearance between plates 116, 118 in the sagittal plane S. For the example of the disk prosthesis 100, herein described by way of example, the angles A are about ten degrees or less but may be larger as desired for a particular use without deviating from the teaching of et present invention. Such dimensioning makes it possible to provide a disk prosthesis with controlled clearances that correspond substantially to the natural movements of the spinal disk.

In the example illustrated with reference to FIGS. 5-9, the spherical cap 148 and the spherical cup 152 are created on the inserts 146,150 attached to the plates 116, 118. Alternatively, and as illustrated with reference to FIGS. 10 and 11, an alternate embodiment includes the spherical cap 148 as an integral part of the second plate 118, the upper plate in the example illustrated. By way of example, for one such embodiment, the spherical cap 148 may be made of titanium, while the spherical cup 152 is arranged in an insert 146 made of ceramic or polyethylene.

Further, in the preceding examples, the spherical cap 148 is carried on the second (upper) plate 118 with the spherical cup 152 carried by the first (lower) plate 116. Alternatively, and with reference to FIGS. 12 and 13, another embodiment may include the spherical cup 152 carried by the second (upper) plate 118, with the spherical cap 148 carried by the first (lower) plate 116. Yet further and by way of example, one embodiment may include a zirconia-on-alumina articulating surface for the spherical cap 148 and the spherical cup 152 of the ball and socket joint 144.

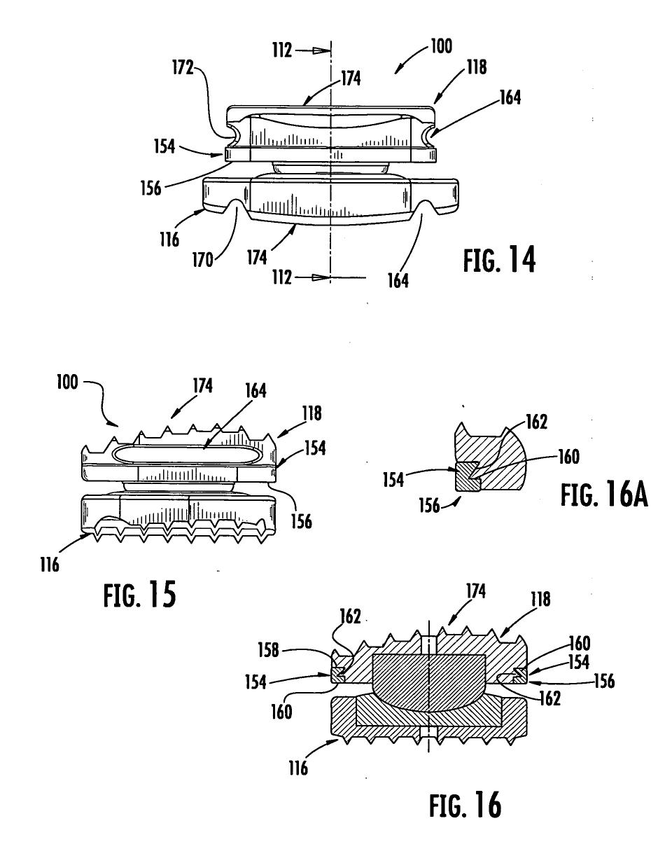

Yet further, and with reference to FIGS. 14-19, by way of example, either the first plate 116 or the second plate 118 may carry a shock absorbing material 154 for contacting the opposing plate 116, 118 at the limits of movement for the articulating plates. As herein presented by way of example, the absorbing material may be an elastic ring 156 extending about and carried in frictional contact within the perimeter portions of the inner faces 124, 126 of the plates 116, 118. As herein illustrated by way of example, the elastic ring 162 may be mounted in a recess 164 surrounding the spherical cap 148 and/or the spherical cup 152 and arranged on the inner face 124, 126. In one embodiment, the plates 116, 118 may include a notch or recess 158 extending about the perimeter fro receiving the elastic ring 156 therein. Yet further, and as illustrated with reference to FIGS. 16 and 16A, the elastic ring 156 may include an inner groove 160 for receiving a tab 162 within a wall of the recess 158 for retaining the elastic ring 156 within the recess 158.

As illustrated with reference again to FIGS. 5 and 8, one embodiment of the discal prosthesis 100 may comprise positioning cutouts 164, such as the holes 166 within the anterior surface of the first plate 116 and holes 168 carried within the second plate 118 for receiving the disk prosthesis holder 400, an instrument for simultaneously holding the plates 116, 118. Alternatively, and as illustrated with reference to FIGS. 14, 15, 17, and 18, the cutouts 164 may comprise a pair of parallel groves 170, 172 carried by each of the first and second plates 116, 118 respectively, for receiving the disk prosthesis holder 400. By way of further example, and with continued reference to FIGS. 14-19, the pairs of parallel groves 170, 172 for the first and second plates 116, 118 may extend from the anterior to the posterior portions thereof, with the first plate including the grooves 170 along the outer face 120 and the second plate 118 including the grooves 172 along opposing side wall surfaces thereof. Such will be the subject of the following description for one embodiment of the disk prosthesis holder 400, but it is to be understood that various modification may be may that are within the scope of the teaching herein presented.

For the above described embodiments of the disk prosthesis 100, the outer faces 120, 122 for at least one of the first and second plates 116, 118 comprises a plurality of teeth 174 for retaining the plates within the adjacent vertebrae 20, 22. As illustrated by way of example with reference again to FIG. 9, at least a portion of the plurality of teeth 174 may comprise an anterior slope bias for facilitating an insertion into the cavity 14 made within the intervertebral space 18 while restricting the retracting of the disk prosthesis 100, as above described with reference to FIGS. 1-4.

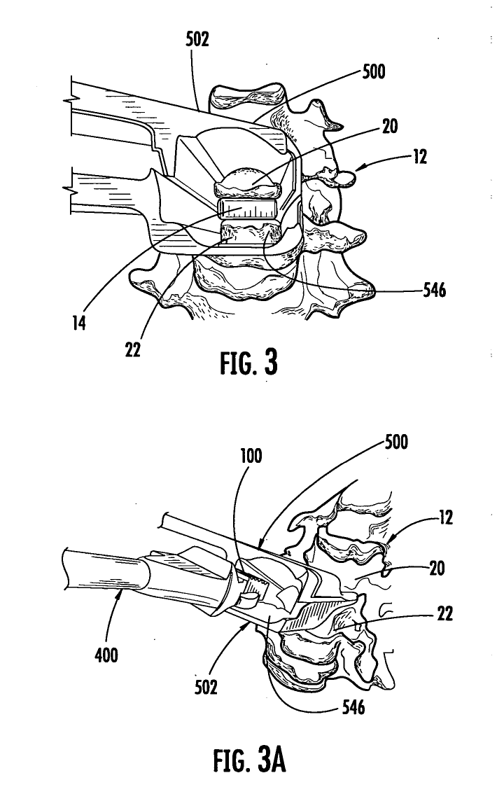

With referenced again to FIGS. 2 and 3, the flat distractor 200 may include a peripheral shape sized for approximating a peripheral shape of the disk prosthesis 100 to be implanted within the intervertebral space 18. The flat distractor 200 is sized for inserting into the cavity 14 formed by removing at least a portion of disk material 24 within the intervertebral disk 16 sufficient for receiving the flat distractor for sizing the cavity. Generally, lateral and facial X-ray images of the flat distractor 200 within the intervertebral space 18 will be obtained for determining a disk alignment 26 line for the intervertebral space. With continued reference to FIGS. 2 and 3, and as illustrated with reference to FIG. 20, the flat distractor 200 may comprise a handle 202 having a proximal end 204 and an opposing distal end 206. A head 208 having an anterior wall 210, an opposing generally flat posterior wall 212, and tapered side walls 214 therebetween is carried at the distal end 206 of the handle 202. For the example herein presented, the anterior wall 210 is fixedly attached to the handle distal end 206. Further, corner portions 216 are formed by the opposing anterior and posterior walls 210, 212 with adjoining side walls 214 having complex contoured surfaces 218 for facilitating movement into and out of the intervertebral space 18.

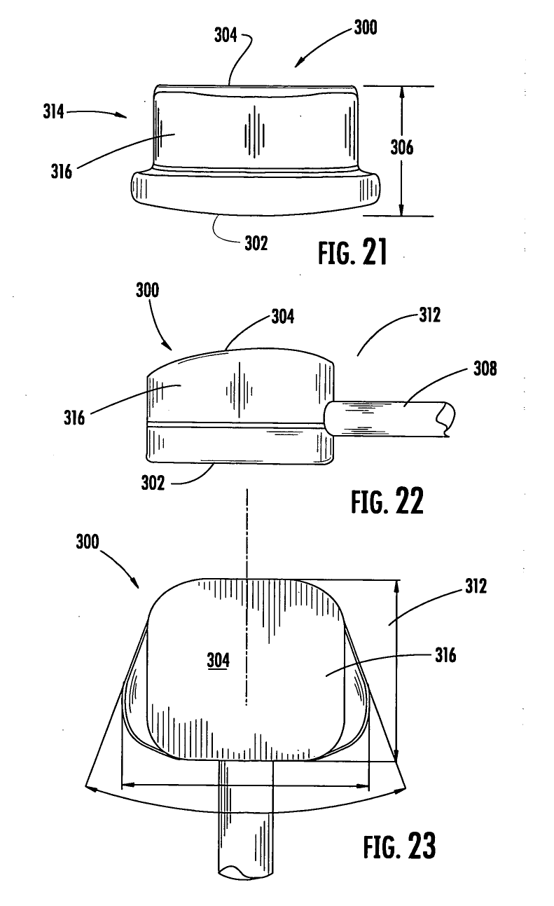

As illustrated with reference again to FIGS. 4 and 4A, and to FIGS. 21-23, the trial implant 300 may have opposing first and second outer surfaces 302, 304, with the trial implant generally comprising an outer shell having a shape comparable to an outer shell of the disk prosthesis 100, as above described. The opposing first and second outer surfaces 302, 304 of the trial implant 300 are generally smooth for facilitating the inserting and retracting into and out of the cavity 14 within the intervertebral space 18 as guided by the disk alignment line 26. The trial implant 300 further comprises a height dimension 306 defined from the first outer surface 302 to the second outer surface 304 equal to a height dimension of the disk prosthesis 100 absent the portion comprising the teeth 174. With continued reference to FIGS. 4 and 4A, one embodiment of the trail implant 300, may include a handle 308 having a proximal end 310 and an opposing distal end 312. A head 314 may have the upper and lower portions 316, 318, wherein a transversal dimension of the lower portion is greater than a corresponding transversal dimension of the upper portion so as to form opposing transverse shelf portions 320, 322 on the lower portion. The head 314 includes the outer face 302 on the lower portion 316 for contacting one of the first and second vertebrae 22 that is larger in surface area than the outer face 304 of the upper portion 318 for contacting another of the first and second vertebrae 20. By way of example for the disk prosthesis 100 and the trial implant 300, herein described by way of example, the lower portion 318 of the trial implant 300 may have a substantially trapezoidal shape in a horizontal plane, with a large base 324 of the lower portion defining a leading edge and a small base 326 thereof defining a trailing edge for the trial implant. The upper portion 316 may have a parallelepiped shape in a horizontal plane as the disk prosthesis 100 with which it is cooperating within the system 10. Yet further, the outer surface 302 of the lower portion 318 may have a convex profile in the frontal plane.

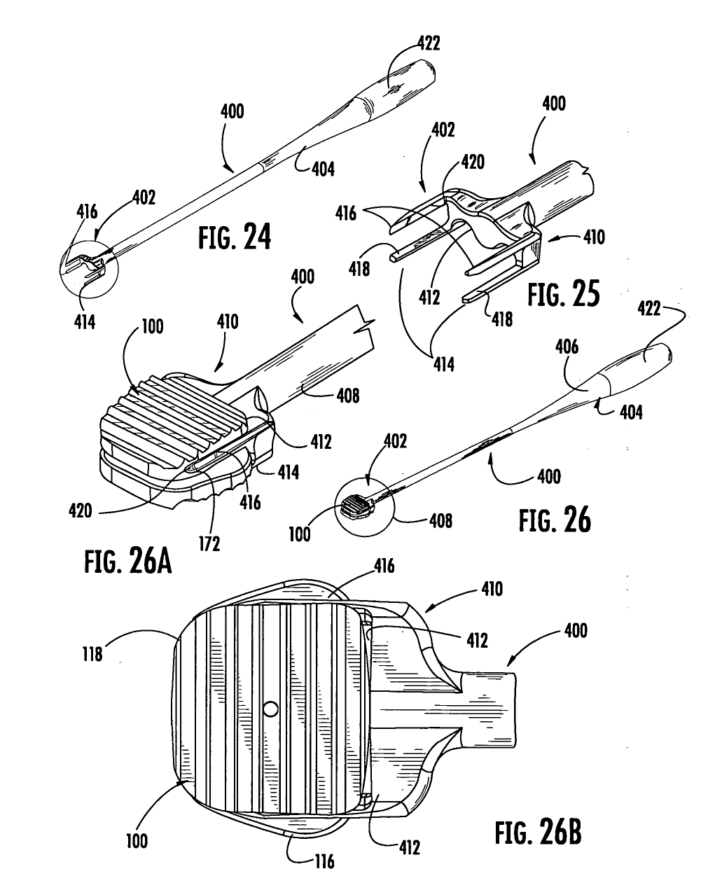

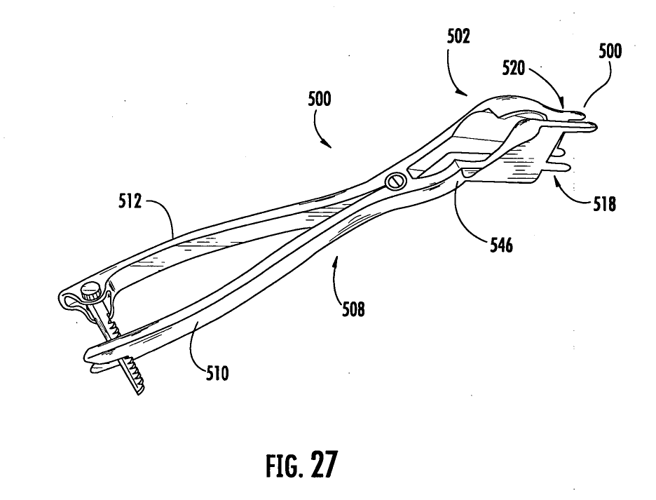

As illustrated with reference again to FIG. 6, and to FIGS. 24-26, the disk prosthesis holder 400 may include a plurality of tines 402 for frictionally engaging the disk prosthesis 100 for securing the disk prosthesis to the disk prosthesis holder. By way of example for the embodiments herein described, each of the plurality of tines 402 are generally parallel for engaging the cutout 164 within the disk prosthesis 100, described earlier with reference to FIGS. 14-19, by way of example, through an anterior to posterior movement therebetween. With continued reference to FIGS. 24-26, the disk prosthesis holder 400 may comprise a handle 404 having a proximal end 406 and a distal end 408. A head 410 includes a base 412 and the plurality of tines 402 extending from the base. The base 412 is biased against the anterior surface wall of the disk prosthesis 100 during the inserting of the disk prosthesis into the cavity 14 formed within the intervertebral space 18, as earlier described.

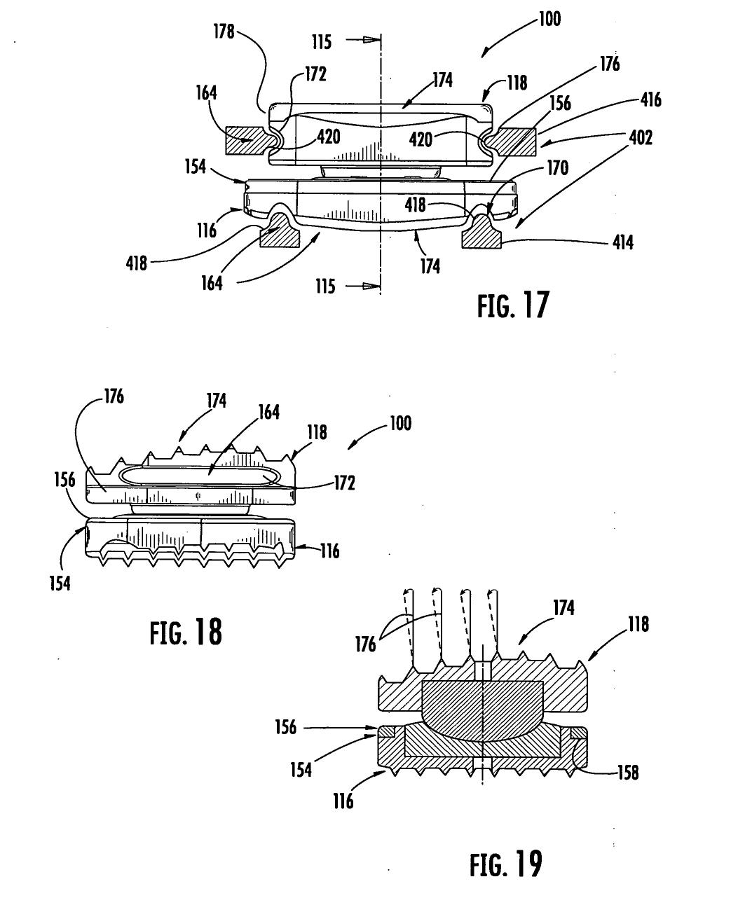

By way of example for the disk prosthesis 100 earlier described, the plurality of tines 402 may comprise a first pair of tines 414 for engaging the first plate 116 of the disk prosthesis and an opposing second pair of tines 416 for engaging the second plate 118 of the disk prosthesis, described with reference to FIGS. 14-19, by way of example. For one embodiment, each of the first pair of tines 414 may comprise a horizontal inner surface 418 for slidably engaging the outer face 120 of the first plate 116. Further, each of the second pair of tines 416 may comprise a vertical inner surface 420 for slidably engaging opposing side wall surfaces 176,178 of the second plate 118, as illustrated by way of example with reference to FIGS. 17 and 18. For the example herein illustrated, the disk prosthesis 100 includes grooves 170, 172 for receiving the tines 414, 416, respectively. However, as will come to the mind of those skilled in the art, the tines 402 may engage the holes 166, 168 earlier described with reference earlier to FIGS. 5 and 8, or without cutouts 164, as herein described. With reference again to FIG. 24, the disk prosthesis holder 400 may include a grip 422 carried by the proximal end 406 of the handle 404.

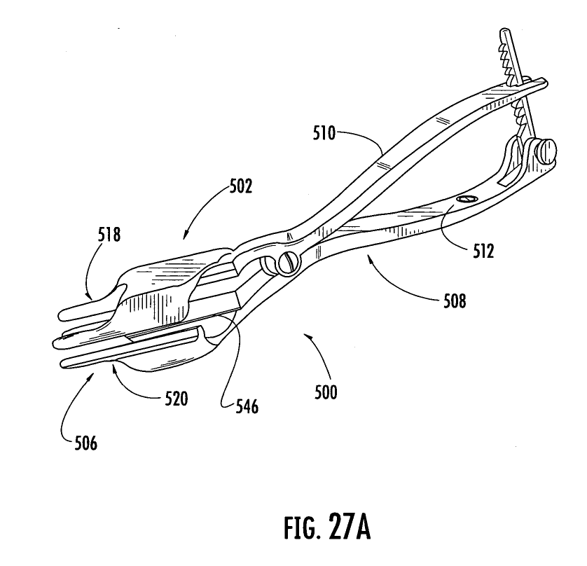

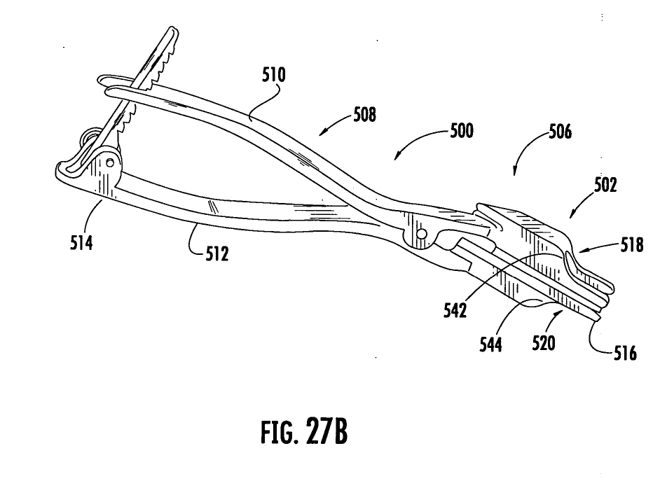

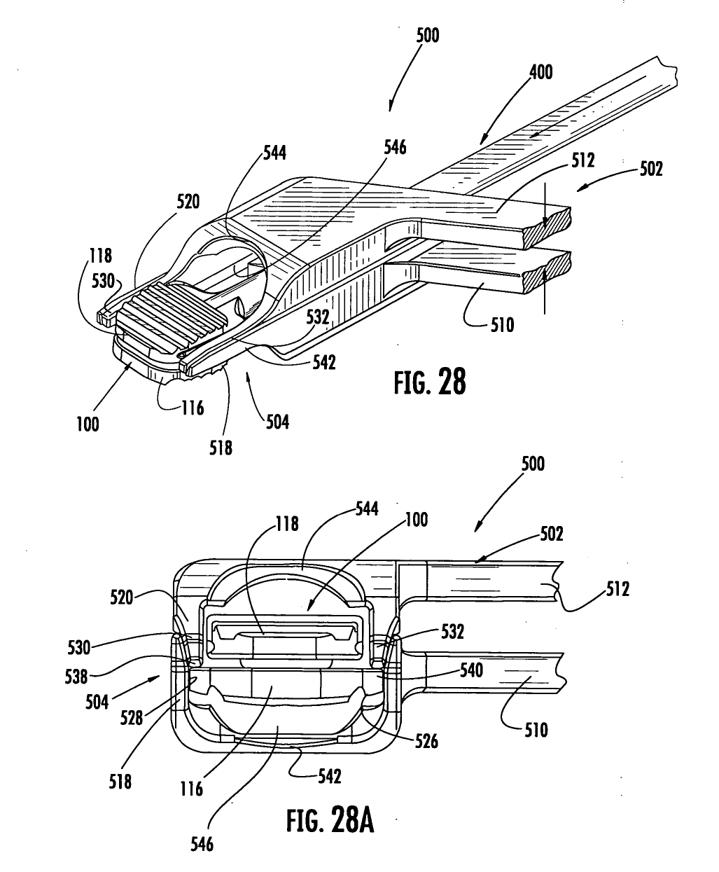

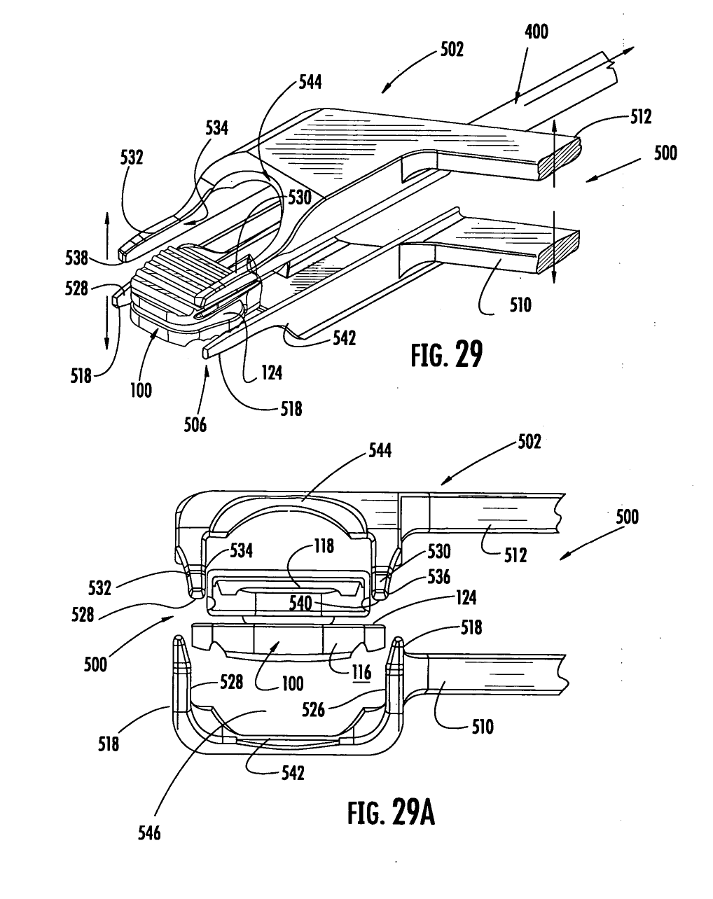

With reference again to FIGS. 5 and 6, and to FIGS. 27, 27A, 27B, 28, 28A, 29, and 29A, one embodiment of the cervical disk distractor 500 may comprise a handle 508, such as forceps by way of example, having first and second arms 510, 512 forming a proximal end 514 and an opposing distal end 516 for the handle, wherein the first and second arms are pivotally attached therebetween. The jaws 502 carried at the distal end 516 of the handle 516 are operable through a leveraging movement of the handle proximal end, as is typical of forceps. With continued reference to FIGS. 28 and 29, and related drawings therefor, the jaws 502 comprise opposing first and second finger element pairs 518, 520, wherein the first finger element pair 518 has transversely opposing finger elements 522, 524 each having an inner surface 526, 528 for slidably receiving the first plate 116 of the disk prosthesis 100 therebetween. For the embodiment herein described by way of example, the second finger element pair 520 includes transversely opposing second finger elements 530, 532, each having an inner surface 534, 536 for slidably receiving the second plate 118 therebetween. The second finger elements 530, 532 each further having a bottom surface 538, 540 for slidably engaging the inner face 124 of the first plate 116 when the jaws 502 are in the closed position 504.

With continued reference to FIGS. 28 and 29, the jaws 502 may comprise opposing base portions 542, 544, each attached to one arm 510, 512 distal end 516, wherein the opposing finger element pairs 518, 520 are carried by the opposing base portions, and wherein a channel 546 is formed by the base portions and finger elements in combination. The channel 546 extends from an anterior end 548 to a posterior end 550, which posterior end defines a tip 552 of the cervical disk distractor 500. The disk holder 400 may then enter the channel 546 from the anterior end 548 when inserting the disk prosthesis 100 into the cavity 14 of the intervertebral space 18. Once the disk prosthesis 100 is desirably in place, the disk distractor 500 is moved to the closed position 504 for having the finger element pairs 518, 520 act as guide rails for closely receiving the disk prosthesis when in the closed position, wherein inserting the tip 552 of the cervical distractor 500 while in the closed position into the intervertebral space 18 when aligning the cervical distractor with the disk alignment line 26 permits opening the cervical distractor for movement from the closed position 504 to the open position 506 for increasing a separation between the first and second vertebrae 16, 18 and providing a physiological opening therefor. Thus, inserting the disk prosthesis 100 between the jaws 502 of the cervical distractor 500 within the channel 546 in the open position using the cervical disk holder 400 allows for an efficient placing of the disk prosthesis 100 within the cavity 14 of the intervertebral space 18 with an aligning of the disk prosthesis using the disk alignment line 26. Closing the jaws 502 permits a removing of the cervical disk holder 400 from the disk prosthesis 100 by pulling the holder away from the disk prosthesis 100. A subsequent removing of the cervical distractor 500 results in the desired implanting of the disk prosthesis 100 within the intervertebral space 18 between the first and second vertebrae 20, 22.

With the above system 10, one method of implanting the disk prosthesis 100 within a cervical spine of a patient may comprise providing access to the intervertebral space 18 between selected vertebrae 20, 22. Using the flat distractor 200 having a shape approximating a shape of the disk prosthesis 100, and removing at least a portion of disk material 24 within the intervertebral space 18 for forming the cavity 14 sufficient for receiving the flat distractor. The flat distractor 200 may be inserted into the cavity 14 multiple times between the removing of portions of the disk material, and until a desirable cavity is formed. Lateral and facial X-ray images may be taken of the flat distractor within the intervertebral space for determining a disk alignment line for the intervertebral space. As above described, the trial implant 300 having opposing first and second outer surfaces 302, 304 and an outer shell having a shape comparable to an outer shell of the disk prosthesis 100, unlike the disk prosthesis has smooth outer surfaces and a height dimension equal to a height dimension of a disk prosthesis to be implanted. As such, the trial implant 300 may be inserted into the cavity 14 and easily removed. Once a desired cavity 14 has been formed and confirmed using the trial implant 300, the tip 552 of the cervical distractor 500, while in the closed position 504, is inserted into the intervertebral space 18 using the disk alignment line 26 as a reference. When in place between the vertebrae 20, 22, the cervical distractor 500 is moved to the open position 506 for increasing a separation between the first and second vertebrae for providing a physiological opening. The disk prosthesis holder 400 frictionally engages the disk prosthesis 100 using the plurality of tines 402 for inserting the disk prosthesis between the jaws 502 of the cervical distractor 500 when in the open position 506 and positions the disk prosthesis 100 within the cavity 14. The disk alignment line 26 may be used to align the disk prosthesis 100. The jaws 502 are then closed and the cervical disk holder 400 is pulled from the intervertebral space 18 leaving the disk prosthesis 100 in place. The cervical distractor 500 is then removed.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that modifications and alternate embodiments are intended to be included within the scope of the appended claims.

That which is claimed:

1. A system for implanting a disk prosthesis within a cervical spine of a patient, the system comprising:
a disk prosthesis having opposing first and second plates and a ball and socket joint interposed therebetween, wherein an outer face for at least one of the first and second plates include a retaining element for retaining the disk prosthesis between adjacent first and second vertebrae of the cervical spine, and wherein at least a portion of the retaining element has an anterior slope bias for facilitating an insertion of the disk prosthesis into an intervertebral space between the adjacent first and second vertebrae while restricting anterior distraction thereof;
a flat distractor having a peripheral shape sized for approximating a peripheral shape of the disk prosthesis to be implanted within the intervertebral space, wherein the flat distractor is sized for inserting into a cavity formed by removing at least a portion of disk material within the intervertebral space sufficient for receiving the flat distractor for sizing the cavity using lateral and facial X-ray images of the flat distractor within the intervertebral space for determining a disk alignment line for the intervertebral space;
a trial implant having opposing first and second outer surfaces, wherein the trial implant generally comprises an outer shell having a shape comparable to an outer shell of the disk prosthesis, and wherein the opposing first and second outer surfaces of the trial implant are generally smooth for facilitating an inserting and detracting thereof into and out of the cavity within the intervertebral space as guided by the disk alignment line for a positioning therein, wherein the trial implant further comprises a height dimension defined from the first to the second outer surfaces equal to a height dimension of the disk prosthesis;
a disk prosthesis holder having a plurality of tines for engaging the disk prosthesis therewith by a frictionally securing thereof, wherein each of the plurality of tines are generally parallel for engaging a cutout within the disk prosthesis through an anterior to posterior movement therebetween;
a cervical disk distractor having jaws operable between a closed position and an open position thereof, the jaws including opposing finger element pairs for freely receiving the disk prosthesis therebetween when in the open position and for providing guide rails closely receiving the disk prosthesis when in the closed position, wherein inserting a tip portion of the cervical distractor while in the closed position into the intervertebral space when aligning the cervical distractor with the disk alignment line permits opening the cervical distractor for movement from the closed position to the open position for increasing a separation between the first and second vertebrae and providing a physiological opening therefor, and wherein inserting the disk prosthesis between the jaws of the cervical distractor in the open position with the cervical disk holder allows for a placing of the disk prosthesis within the cavity of the intervertebral space with an aligning of the disk prosthesis using the disk alignment line, and wherein closing the jaws of the cervical distractor permits a removing of the cervical disk holder from the disk prosthesis by pulling the holder therefrom with a subsequent removing of the cervical distractor resulting in an implanting of the disk prosthesis within the intervertebral space between the first and second vertebrae.

2. A system according to claim 1, wherein the ball and socket joint of the disk prosthesis is interposed between the first and second plates stacked one on top of the other such that inner faces of the plates are turned toward one another, and wherein a ball portion of the ball and socket joint includes a spherical cap cooperating with a socket portion of the joint including a spherical cup.

3. A system according to claim 1, wherein the disk prosthesis includes a transversal dimension of the first plate greater than a corresponding dimension of the second plate so as to constitute a limit stop by the inner face of the first plate for the second plate, and a bearing surface of the outer face of the first plate for the vertebrae that is larger than a bearing surface for the outer face of the second plate.

4. A system according to claim 1, wherein the first plate has a substantially trapezoidal shape in a horizontal plane, wherein a large base of the first plate defines a leading edge and a small base thereof defines a trailing edge for the disk prosthesis.

5. A system according to claim 1, wherein the second plate has a parallelepiped shape in a horizontal plane.

6. A system according to claim 1, wherein the first plate has an inner face with a flat profile in a frontal plane and an outer face with a convex profile in the frontal plane.

7. A system according to claim 1, wherein the cutout in the disk prosthesis comprises at least one cutout carried within each of the first and second plates for receiving the plurality of tines of the disk prosthesis holder for simultaneously holding the plates in a fixed position.

8. A system according claim 7, wherein the at least one cutout comprises a hole carried within an anterior portion of each of the first and second plates.

9. A system according to claim 7, wherein at least one cutout for each of the first and second plates comprises a pair of parallel grooves carried by each of the first and second plates for receiving the plurality of tines therein.

10. A system according to claim 9, wherein the pair of parallel grooves for the first and second plates extend from anterior toward a posterior portion thereof, and wherein the first plate includes the grooves along the outer face thereof and the second plate includes the grooves along opposing side wall surfaces thereof.

11. A system according to claim 1, wherein the retaining element comprises a plurality of teeth.

12. A system according to claim 1, wherein the flat distractor further comprises:
a handle having a proximal end and an opposing distal end; and
a head having an anterior wall, an opposing generally flat posterior wall, and tapered side walls therebetween, wherein the anterior wall is fixedly attached to the handle distal end.

13. A system according to claim 12, wherein corner portions formed by the opposing anterior and posterior walls with adjoining side walls have complex contoured surfaces for facilitating movement into and out of the intervertebral space.

14. A system according 1, wherein the trial further implant comprises:
a handle having a proximal end and an opposing distal end; and
a head having upper and lower portion thereof, wherein a transversal dimension of the lower portion is greater than a corresponding transversal dimension of the upper portion so as to form opposing transverse shelf portions on the lower portion, the head having an outer face on the lower portion for contacting one of the first and second vertebrae that is larger than an outer face of the upper portion for contacting another of the first and second vertebrae.

15. A system according to claim 14, wherein the lower portion of the trial implant has a substantially trapezoidal shape in a horizontal plane, with a large base of the lower portion defining a leading edge and a small base thereof defining a trailing edge for the trial implant.

16. A system according to claim 15, wherein the upper portion of the trial implant has a parallelepiped shape in a horizontal plane.

17. A system according to claim 14, wherein the outer face of the lower portion has a convex profile in the frontal plane.

18. A system according to claim 1, the disk prosthesis holder further comprises:
a handle having a proximal end and a distal end; and
a head having a base and the plurality of tines extending therefrom, wherein the base is biased against the anterior surface wall of the disk prosthesis during the inserting of the disk prosthesis into the intervertebral space.

19. A system according to claim 18, wherein the plurality of tines comprises a first pair of tines for engaging the first plate of the disk prosthesis and an opposing second pair of tines for engaging the second plate of the disk prosthesis.

20. A system according to claim 18, wherein each of the first pair of tines comprise a horizontal inner surface for slidably engaging the outer face of the first plate, and wherein each of the second pair of tines comprise a vertical inner surface for slidably engaging opposing side wall surfaces of the second plate.

21. A system according to claim 18, wherein the handle proximal end comprises a grip portion.

22. A system according to claim 1, wherein the cervical disk distractor further comprises:
a handle having first and second arms each having a proximal end and an opposing distal end, wherein the first and second arms are pivotally attached therebetween;
the jaws carried at the distal end of the arm for operation thereof through leveraging movement of the handle proximal end.

23. A system according to claim 22, wherein the opposing finger element pairs of the jaws comprises:
a first finger element pair having transversely opposing finger elements each having an inner surface for slidably receiving the first plate of the disk prosthesis therebetween;
a second finger element pair having transversely opposing finger elements each having an inner surface for slidably receiving the second plate therebetween, the second finger elements each further having a bottom surface for slidably engaging the inner surface of the first plate when the jaws are in the closed position.

24. A system according to claim 22, wherein the jaws are comprise opposing base portions each attached to one arm distal end, wherein the opposing finger element pairs are carried by the opposing base portions, and wherein a channel by the base portions and finger elements, which channel extends from an anterior end to a posterior end defining the tip portion of the cervical disk distractor and wherein the disk holder enters the channel from the anterior end when inserting the disk prosthesis into the cavity of the intervertebral space.

25. A system for implanting a disk prosthesis within a cervical spine of a patient, the system comprising:
a disk prosthesis having opposing first and second plates and a ball and socket joint interposed therebetween, wherein an outer face for at least one of the first and second plates include a retaining element for retaining the disk prosthesis between adjacent first and second vertebrae of the cervical spine;
a disk prosthesis holder having a plurality of tines for engaging the disk prosthesis therewith, wherein each of the plurality of tines are dimensioned for engaging a cutout within the disk prosthesis;
a cervical disk distractor having jaws operable between a closed position and an open position thereof, the jaws including opposing element pairs for freely receiving the disk prosthesis therebetween when in the open position and for providing guide rails closely receiving the disk prosthesis when in the closed position, wherein inserting a tip portion of the cervical distractor while in the closed position into the intervertebral space permits opening the cervical distractor for movement from the closed position to the open position for increasing a separation between the first and second vertebrae, and wherein inserting the disk prosthesis between the jaws of the cervical distractor in the open position with the cervical disk holder allows for a placing of the disk prosthesis within the cavity of an intervertebral space, and wherein closing the jaws of the cervical distractor permits a removing of the cervical disk holder from the disk prosthesis by pulling the holder therefrom with a subsequent removing of the cervical distractor resulting in an implanting of the disk prosthesis within the intervertebral space between the first and second vertebrae.

26. A system according to claim 25, further comprising a flat distractor having a peripheral shape sized for approximating a peripheral shape of the disk prosthesis to be implanted within the intervertebral space, wherein the flat distractor is sized for inserting into a cavity formed by removing at least a portion of disk material within the intervertebral space sufficient for receiving the flat distractor for sizing the cavity.

27. A system according to claim 26, wherein the cavity sizing includes use of lateral and facial X-ray images of the flat distractor within the intervertebral space for determining a disk alignment line for the intervertebral space;

28. A system according to claim 25, further comprising a trial implant having opposing first and second outer surfaces, wherein the trial implant generally comprises an outer shell having a shape comparable to an outer shell of the disk prosthesis, and wherein the opposing first and second outer surfaces of the trial implant are generally smooth for facilitating an inserting and detracting thereof into and out of the cavity within the intervertebral space, and wherein the trial implant has a height dimension defined from the first to the second outer surfaces equal to a height dimension of the disk prosthesis.

29. A system according to claim 25, wherein at least a portion of the retaining element has an anterior slope bias for facilitating an insertion of the disk prosthesis into the intervertebral space between the adjacent first and second vertebrae while restricting anterior distraction thereof.

30. A system according to claim 25, wherein the ball and socket joint of the disk prosthesis is interposed between the first and second plates stacked one on top of the other such that inner faces of the plates are turned toward one another, and wherein a ball portion of the ball and socket joint includes a spherical cap cooperating with a socket portion of the joint including a spherical cup.

31. A system according to claim 25, wherein the disk prosthesis includes a transversal dimension of the first plate greater than a corresponding dimension of the second plate so as to constitute a limit stop by the inner face of the first plate for the second plate, and a bearing surface of the outer face of the first plate for the vertebrae that is larger than a bearing surface for the outer face of the second plate.

32. A system according to claim 25, wherein the first plate has a substantially trapezoidal shape in a horizontal plane, wherein a large base of the first plate defines a leading edge and a small base thereof defines a trailing edge for the disk prosthesis.

33. A system according to claim 25, wherein the second plate has a parallelepiped shape in a horizontal plane.

34. A system according to claim 25, wherein the first plate has an inner face with a flat profile in a frontal plane and an outer face with a convex profile in the frontal plane.

35. A system according to claim 25, wherein the cutout in the disk prosthesis comprises at least one cutout carried within each of the first and second plates for receiving the plurality of tines of the disk prosthesis holder for simultaneously holding the plates in a fixed position.

36. A system according claim 35, wherein the at least one cutout comprises a hole carried within an anterior portion of each of the first and second plates.

37. A system according to claim 35, wherein at least one cutout for each of the first and second plates comprises a pair of parallel grooves carried by each of the first and second plates for receiving the plurality of tines therein.

38. A system according to claim 37, wherein the pair of parallel grooves for the first and second plates extend from anterior toward a posterior portion thereof, and wherein the first plate includes the grooves along the outer face thereof and the second plate includes the grooves along opposing side wall surfaces thereof.

39. A system according to claim 25, wherein the retaining element comprises a plurality of teeth.

40. A system according to claim 25, the disk prosthesis holder further comprises:
a handle having a proximal end and a distal end; and
a head having a base and the plurality of tines extending therefrom,
wherein the base is biased against the anterior surface wall of the disk prosthesis during the inserting of the disk prosthesis into the intervertebral space.

41. A system according to claim 40, wherein the plurality of tines comprises a first pair of tines for engaging the first plate of the disk prosthesis and an opposing second pair of tines for engaging the second plate of the disk prosthesis.

42. A system according to claim 40, wherein each of the first pair of tines comprise a horizontal inner surface for slidably engaging the outer face of the first plate, and wherein each of the second pair of tines comprise a vertical inner surface for slidably engaging opposing side wall surfaces of the second plate.

43. A system according to claim 25, wherein the cervical disk distractor further comprises:
a handle having first and second arms each having a proximal end and an opposing distal end, wherein the first and second arms are pivotally attached therebetween;
the jaws carried at the distal end of the arm for operation thereof through leveraging movement of the handle proximal end.

44. A system according to claim 43, wherein the opposing finger element pairs of the jaws comprises:
a first finger element pair having transversely opposing finger elements each having an inner surface for slidably receiving the first plate of the disk prosthesis therebetween;
a second finger element pair having transversely opposing finger elements each having an inner surface for slidably receiving the second plate therebetween, the second finger elements each further having a bottom surface for slidably engaging the inner surface of the first plate when the jaws are in the closed position.

45. A system according to claim 43, wherein the jaws are comprise opposing base portions each attached to one arm distal end, wherein the opposing finger element pairs are carried by the opposing base portions, and wherein a channel by the base portions and finger elements, which channel extends from an anterior end to a posterior end defining the tip portion of the cervical disk distractor and wherein the disk holder enters the channel from the anterior end when inserting the disk prosthesis into the cavity of the intervertebral space.

46. A system for implanting a disk prosthesis within a cervical spine of a patient, the system comprising:
a disk prosthesis having opposing first and second plates and a ball and socket joint interposed therebetween, wherein an outer face for at least one of the first and second plates include a retaining element for retaining the disk prosthesis between adjacent first and second vertebrae of the cervical spine; and
a disk prosthesis holder having a plurality of tines for engaging the disk prosthesis therewith, wherein each of the plurality of tines are dimensioned for engaging a cutout within the disk prosthesis.

47. A system according to claim 46, further comprising a cervical disk distractor having jaws operable between a closed position and an open position thereof, the jaws including opposing element pairs for freely receiving the disk prosthesis therebetween when in the open position and for providing guide rails closely receiving the disk prosthesis when in the closed position, wherein inserting a tip portion of the cervical distractor while in the closed position into the intervertebral space permits opening the cervical distractor for movement from the closed position to the open position for increasing a separation between the first and second vertebrae, and wherein inserting the disk prosthesis between the jaws of the cervical distractor in the open position with the cervical disk holder allows for a placing of the disk prosthesis within the cavity of an intervertebral space, and wherein closing the jaws of the cervical distractor permits a removing of the cervical disk holder from the disk prosthesis by pulling the holder therefrom with a subsequent removing of the cervical distractor resulting in an implanting of the disk prosthesis within the intervertebral space between the first and second vertebrae.

48. A system according to claim 46, further comprising a flat distractor having a peripheral shape sized for approximating a peripheral shape of the disk prosthesis to be implanted within the intervertebral space, wherein the flat distractor is sized for inserting into a cavity formed by removing at least a portion of disk material within the intervertebral space sufficient for receiving the flat distractor for sizing the cavity.

49. A system according to claim 46, further comprising a trial implant having opposing first and second outer surfaces, wherein the trial implant generally comprises an outer shell having a shape comparable to an outer shell of the disk prosthesis, and wherein the opposing first and second outer surfaces of the trial implant are generally smooth for facilitating an inserting and detracting thereof into and out of the cavity within the intervertebral space, and wherein the trial implant has a height dimension defined from the first to the second outer surfaces equal to a height dimension of the disk prosthesis.

50. A system according to claim 46, wherein at least a portion of the retaining element has an anterior slope bias for facilitating an insertion of the disk prosthesis into the intervertebral space between the adjacent first and second vertebrae while restricting anterior distraction thereof.

51. A system according to claim 46, wherein the ball and socket joint of the disk prosthesis is interposed between the first and second plates stacked one on top of the other such that inner faces of the plates are turned toward one another, and wherein a ball portion of the ball and socket joint includes a spherical cap cooperating with a socket portion of the joint including a spherical cup.

52. A system according to claim 46, wherein the disk prosthesis includes a transversal dimension of the first plate greater than a corresponding dimension of the second plate so as to constitute a limit stop by the inner face of the first plate for the second plate, and a bearing surface of the outer face of the first plate for the vertebrae that is larger than a bearing surface for the outer face of the second plate.

53. A system according to claim 46, wherein the first plate has a substantially trapezoidal shape in a horizontal plane, wherein a large base of the first plate defines a leading edge and a small base thereof defines a trailing edge for the disk prosthesis.

54. A system according to claim 46, wherein the second plate has a parallelepiped shape in a horizontal plane.

55. A system according to claim 46, wherein the first plate has an inner face with a flat profile in a frontal plane and an outer face with a convex profile in the frontal plane.

56. A system according to claim 46, wherein the cutout in the disk prosthesis comprises at least one cutout carried within each of the first and second plates for receiving the plurality of tines of the disk prosthesis holder for simultaneously holding the plates in a fixed position.

57. A system according claim 56, wherein the at least one cutout comprises a hole carried within an anterior portion of each of the first and second plates.

58. A system according to claim 56, wherein at least one cutout for each of the first and second plates comprises a pair of parallel grooves carried by each of the first and second plates for receiving the plurality of tines therein.

59. A system according to claim 58, wherein the pair of parallel grooves for the first and second plates extend from anterior toward a posterior portion thereof, and wherein the first plate includes the grooves along the outer face thereof and the second plate includes the grooves along opposing side wall surfaces thereof.

60. A system according to claim 46, wherein the retaining element comprises a plurality of teeth.

61. A system according to claim 46, the disk prosthesis holder further comprises:
a handle having a proximal end and a distal end; and
a head having a base and the plurality of tines extending therefrom,
wherein the base is biased against the anterior surface wall of the disk prosthesis during the inserting of the disk prosthesis into the intervertebral space.

62. A system according to claim 61, wherein the plurality of tines comprises a first pair of tines for engaging the first plate of the disk prosthesis and an opposing second pair of tines for engaging the second plate of the disk prosthesis.

63. A system according to claim 62, wherein each of the first pair of tines comprise a horizontal inner surface for slidably engaging the outer face of the first plate, and wherein each of the second pair of tines comprise a vertical inner surface for slidably engaging opposing side wall surfaces of the second plate.

64. A method of implanting a disk prosthesis within a cervical spine of a patient, the method comprising:
providing access to an intervertebral space between first and second vertebrae;
providing a flat distractor having a shape approximating a shape of a disk prosthesis to be implanted within the intervertebral space;
removing at least a portion of disk material within the intervertebral space for forming a cavity therein sufficient for receiving the flat distractor;
inserting the flat distractor into the cavity for a sizing thereof;
obtaining lateral and facial X-ray images of the flat distractor within the intervertebral space for determining a disk alignment line for the intervertebral space;
providing a trial implant having opposing first and second outer surfaces, wherein the trial implant generally comprises an outer shell having a shape comparable to an outer shell of a disk prosthesis to be implanted in the intervertebral space, and wherein outer surfaces of the trial implant are generally smooth, wherein the trial implant further comprises a height dimension equal to a height dimension of a disk prosthesis to be implanted;
inserting the trial implant into the cavity of the intervertebral space using the disk alignment line for a positioning thereof;
removing the trial implant;
providing a cervical distractor having jaws operable between a closed position and an open position thereof, the jaws including opposing finger element pairs for freely receiving the disk prosthesis therebetween when in the open position and for providing guide rails closely receiving the disk prosthesis when in the closed position;
inserting a tip portion of the cervical distractor while in the closed position into the intervertebral space;
aligning the cervical distractor with the disk alignment line;
opening the cervical distractor for movement from the closed position to the open position for increasing a separation between the first and second vertebrae and providing a physiological opening therefor;
providing a cervical disk holder having a plurality of tines for engaging a disk prosthesis therebetween;
providing a disk prosthesis having opposing first and second plates and a ball and socket joint interposed therebetween, wherein an outer face for at least one of the first and second plates includes a plurality of teeth for retaining the plates between the adjacent vertebrae, and wherein at least a portion of the plurality of teeth have an anterior slope bias for facilitating an insertion of the disk prosthesis into the intervertebral space while restricting anterior distraction thereof;
frictionally securing the disk prosthesis to the cervical disk holder with the plurality of tines, wherein each of the plurality of tines engages a cutout within the disk prosthesis;
inserting the disk prosthesis between the jaws of the cervical distractor in the open position with the cervical disk holder for placing the disk prosthesis within the cavity of the intervertebral space;
aligning the disk prosthesis using the disk alignment line;
closing the jaws of the cervical distractor;
removing the cervical disk holder by pulling the holder from the disk prosthesis; and
removing the cervical distractor.

65. A method of implanting a disk prosthesis within a spine of a patient, the method comprising:
providing access to an intervertebral space between first and second vertebrae;
providing a cervical distractor having jaws operable between a closed position and an open position thereof, the jaws including opposing finger element pairs for freely receiving the disk prosthesis therebetween when in the open position and for providing guide rails closely receiving the disk prosthesis when in the closed position;
inserting a tip portion of the cervical distractor while in the closed position into the intervertebral space;
opening the cervical distractor for movement from the closed position to the open position for increasing a separation between the first and second vertebrae and providing a physiological opening therefor;
providing a cervical disk holder having a plurality of tines for engaging a disk prosthesis therebetween;
providing a disk prosthesis having opposing first and second plates and a ball and socket joint interposed therebetween, wherein an outer face for at least one of the first and second plates includes a plurality of teeth for retaining the plates between the adjacent vertebrae, and wherein at least a portion of the plurality of teeth have an anterior slope bias for facilitating an insertion of the disk prosthesis into the intervertebral space while restricting anterior distraction thereof;
removably securing the disk prosthesis to the cervical disk holder with the plurality of tines, wherein each of the plurality of tines engages a cutout within the disk prosthesis;
inserting the disk prosthesis between the jaws of the cervical distractor in the open position with the cervical disk holder for placing the disk prosthesis within the cavity of the intervertebral space;
closing the jaws of the cervical distractor;
removing the cervical disk holder by pulling the holder from the disk prosthesis; and
removing the cervical distractor.

66. A method according to claim 65 further comprising:
providing a flat distractor having a shape approximating a shape of a disk prosthesis to be implanted within the intervertebral space;
removing at least a portion of disk material within the intervertebral space for forming the cavity therein sufficient for receiving the flat distractor;
inserting the flat distractor into the cavity for a sizing thereof.

67. A method according to claim 66, further comprising obtaining lateral and facial X-ray images of the flat distractor within the intervertebral space for determining a disk alignment line for the intervertebral space.

68. A method according to claim 67, further comprising aligning the cervical distractor with the disk alignment line.

69. A method according to claim 67, further comprising aligning the disk prosthesis using the disk alignment line.

70. A method according to claim 65, further comprising:
providing a trial implant having opposing first and second outer surfaces, wherein the trial implant generally comprises an outer shell having a shape comparable to an outer shell of a disk prosthesis to be implanted in the intervertebral space, and wherein outer surfaces of the trial implant are generally smooth, wherein the trial implant further comprises a height dimension equal to a height dimension of a disk prosthesis to be implanted;
inserting the trial implant into the cavity of the intervertebral space for a positioning therein; and
removing the trial implant.

71. A method of implanting a disk prosthesis within a spine of a patient, the method comprising:
providing access to an intervertebral space between first and second vertebrae;
providing a cervical disk holder having a plurality of tines for engaging a disk prosthesis therebetween;
providing a disk prosthesis having opposing first and second plates and a ball and socket joint interposed therebetween, wherein an outer face for at least one of the first and second plates includes a plurality of teeth for retaining the plates between the adjacent vertebrae, and wherein at least a portion of the plurality of teeth have an anterior slope bias for facilitating an insertion of the disk prosthesis into the intervertebral space while restricting anterior distraction thereof;
removably securing the disk prosthesis to the cervical disk holder with the plurality of tines, wherein each of the plurality of tines engages a cutout within the disk prosthesis;
inserting the disk prosthesis between the jaws of the cervical distractor in the open position with the cervical disk holder for placing the disk prosthesis within the cavity of the intervertebral space; and
removing the cervical disk holder by pulling the holder from the disk prosthesis, the disk prosthesis retained within the intervertebral space by the plurality of teeth and slidably removed from the disk prosthesis holder by the pulling movement thereof.

72. A method according to claim 71, further comprising:
providing a cervical distractor having jaws operable between a closed position and an open position thereof, the jaws including opposing finger element pairs for freely receiving the disk prosthesis therebetween when in the open position and for providing guide rails closely receiving the disk prosthesis when in the closed position;
inserting a tip portion of the cervical distractor while in the closed position into the intervertebral space;
opening the cervical distractor for movement from the closed position to the open position for increasing a separation between the first and second vertebrae and providing a physiological opening therefor;
closing the jaws of the cervical distractor; and
removing the cervical distractor.

73. A method according to claim 71 further comprising:
providing a flat distractor having a shape approximating a shape of a disk prosthesis to be implanted within the intervertebral space;
removing at least a portion of disk material within the intervertebral space for forming the cavity therein sufficient for receiving the flat distractor; and
inserting the flat distractor into the cavity for a sizing thereof.

74. A method according to claim 73, further comprising obtaining lateral and facial X-ray images of the flat distractor within the intervertebral space for determining a disk alignment line for the intervertebral space.

75. A method according to claim 74, further comprising aligning the cervical distractor with the disk alignment line.

76. A method according to claim 74, further comprising aligning the disk prosthesis using the disk alignment line.

77. A method according to claim 71, further comprising:
providing a trial implant having opposing first and second outer surfaces, wherein the trial implant generally comprises an outer shell having a shape comparable to an outer shell of a disk prosthesis to be implanted in the intervertebral space, and wherein outer surfaces of the trial implant are generally smooth, wherein the trial implant further comprises a height dimension equal to a height dimension of a disk prosthesis to be implanted;
inserting the trial implant into the cavity of the intervertebral space for a positioning therein; and
removing the trial implant.
